(19) United States
(12) Patent Application Publication
(10) Pub. No.: US 2019/0337869 A1
(43) Pub. Date: Nov. 7, 2019

(54) SYSTEM AND METHOD FOR PRODUCTION OF CHEMICAL FEEDSTOCK FROM CRUDE OIL

(71) Applicants: UOP LLC, Des Plaines, IL (US); SABIC Global Technologies B.V., Bergen op Zoom (NL)

(72) Inventors: Selman Ziya Erisken, Chicago, IL (US); Heinrich Manfred Schrod, Riyadh (SA)

(21) Appl. No.: 16/311,273

(22) PCT Filed: Jun. 12, 2017
(86) PCT No.: PCT/US2017/036957

§ 371 Date: Dec. 19, 2018

Related U.S. Application Data
(60) Provisional application No. 62/352,729, filed on Jun. 21, 2016.

(51) Int. Cl. C07C 5/03; C10G 69/00; C07C 5/27; C07C 4/04; B01J 19/24; B01J 27/125; B01J 23/883; B01D 3/14

(52) U.S. Cl. CPC C07C 5/03; C10G 69/00; C07C 5/2702; C07C 4/04; C10G 2400/28; B01J 19/245; B01J 27/125; B01J 23/883; B01D 3/14; B01J 19/2445

(57) ABSTRACT

The present disclosure provides a system and method for converting crude oil to light hydrocarbon products useful as a chemical feedstock. The system may also include a conversion system, such as a steam cracking unit, that converts the chemical feedstock from the feed preparation system to useful hydrocarbon chemicals. Exemplary hydrocarbon chemicals produced by the conversion system include light olefins, such as ethylene, propylene, and/or butadiene.

### CROSS REFERENCE TO RELATED APPLICATION

This application is the U.S. national stage of PCT/US2017/036957, published as WO 2017/222850, filed Jun. 12, 2017, which claims the benefit under Title 35, U.S.C. § 119(e) of U.S. Provisional Patent Application Ser. No. 62/352,729, filed Jun. 21, 2016, the disclosures of each of which are hereby incorporated by reference herein in their entirety.

### FIELD OF THE DISCLOSURE

The present disclosure relates to a system and method for refining crude oil. More particularly, the present disclosure relates to a system and method for refining crude oil to produce a chemical feedstock.

### BACKGROUND AND SUMMARY OF THE DISCLOSURE

Conventional oil refineries convert crude oil to transportation fuels, such as gasoline, jet fuel, diesel fuel, fuel oil, and solid fuel. Thus, such conventional oil refineries are generally designed to maximize the production of transportation fuels and to minimize the production of light (i.e., low boiling) hydrocarbon products, such as naphtha, liquefied petroleum gas (LPG), and other light paraffins as well as heavy hydrocarbons such as fuel oil. Naphtha, for example, is usually converted by catalytic reforming to highly aromatic and branched paraffin hydrocarbons useful as high-octane transportation fuel.

The present disclosure provides a refinery configuration that differs from conventional refineries by minimizing production of transportation fuels in favor of light hydrocarbon products useful as a chemical feedstock. The chemical feedstock may then be converted to other useful hydrocarbon chemicals. For example, the chemical feedstock may be fed to a steam cracker unit and converted to light olefins and di-olefins, such as ethylene, propylene, and/or butadiene and C4 olefins.

According to an exemplary embodiment of the present disclosure, a system is provided for converting a crude oil feedstock to a chemical feedstock for a steam cracker unit. The system includes a distillation unit that separates the crude oil feedstock into at least: a hydrocarbon gas fraction; a naphtha fraction; a middle distillate fraction; and a residue fraction; a first isomerization unit having a first input that receives the hydrocarbon gas fraction from the distillation unit and a first output, the first isomerization unit establishing equilibrium by converting a branched butane in the first input to a normal butane in the first output; a naphtha hydrotreating unit that receives the naphtha fraction from the distillation unit, the naphtha hydrotreating unit saturating the naphtha fraction; a middle distillate hydrotreating unit that receives the middle distillate fraction from the distillation unit, the middle distillate hydrotreating unit saturating the middle distillate fraction; a residue hydrotreating unit that receives the residue fraction from the distillation unit, the residue hydrotreating unit saturating the residue fraction; a cracking unit that receives the saturated residue fraction from the residue hydrotreating unit, the cracking unit converting the saturated residue fraction into at least: an unsaturated gas fraction; a light hydrocarbon fraction rich in olefins and aromatics; a light cycle oil fraction rich in rich in multi-ring aromatics; and a heavy waste fraction; a butene processing unit that receives the unsaturated gas fraction from the cracking unit, the butene processing unit producing at least an iso-butane rich fraction; a second isomerization unit having a second input that receives the light hydrocarbon fraction from the cracking unit and a second output, the second isomerization unit establishing equilibrium by converting a branched pentane and a branched hexane in the second input to a normal pentane and a normal hexane in the second output; and an aromatic saturation unit that receives the light cycle oil fraction from the cracking unit, the aromatic saturation unit saturating the light cycle oil fraction and producing a naphthene-rich fraction; and wherein the chemical feedstock to the steam cracker unit includes at least: the first output from the first isomerization unit; the saturated naphtha fraction from the naphtha hydrotreating unit; the saturated middle distillate fraction from the middle distillate hydrotreating unit; the second output from the second isomerization unit; and the naphthene-rich fraction from the aromatic saturation unit.

According to another exemplary embodiment of the present disclosure, a method is provided for converting a crude oil feedstock to a chemical feedstock for a steam cracker unit. The method includes separating the crude oil feedstock into at least: a hydrocarbon gas fraction; a naphtha fraction; a middle distillate fraction; and a residue fraction; converting a branched butane in the hydrocarbon gas fraction to a normal butane in a first isomerization unit by establishing equilibrium in the first isomerization unit; saturating the naphtha fraction in a naphtha hydrotreating unit; saturating the middle distillate fraction in a middle distillate hydrotreating unit; saturating the residue fraction in a residue hydrotreating unit; converting the saturated residue fraction in a cracking unit into at least: an unsaturated gas fraction; a light hydrocarbon fraction rich in olefins and aromatics; a light cycle oil fraction rich in rich in multi-ring aromatics; and a heavy waste fraction; converting the unsaturated gas fraction into at least an iso-butane rich fraction in a butene processing unit; converting a branched pentane and a branched hexane in the light hydrocarbon fraction to a normal pentane and a normal hexane in a second isomerization unit by establishing equilibrium in the second isomerization unit; saturating the light cycle oil fraction in an aromatic saturation unit to produce a naphthene-rich fraction; and directing the chemical feedstock to the steam cracker unit, wherein the chemical feedstock includes at least: the normal butane from the first isomerization unit; the saturated naphtha fraction from the naphtha hydrotreating unit; the saturated middle distillate fraction from the middle distillate hydrotreating unit; the normal pentane and the normal hexane from the second isomerization unit; and the naphthene-rich fraction from the aromatic saturation unit.

### BRIEF DESCRIPTION OF THE DRAWING

The above-mentioned and other features and advantages of this disclosure, and the manner of attaining them, will become more apparent and the invention itself will be better understood by reference to the following description of embodiments of the invention taken in conjunction with the accompanying drawing, wherein:

FIG. 1 is a schematic view of an exemplary system of the present disclosure, the system including a feed preparation system and a conversion system.

The exemplifications set out herein illustrate exemplary embodiments of the invention and such exemplifications are not to be construed as limiting the scope of the invention in any manner.

### DETAILED DESCRIPTION

### 1. System Overview

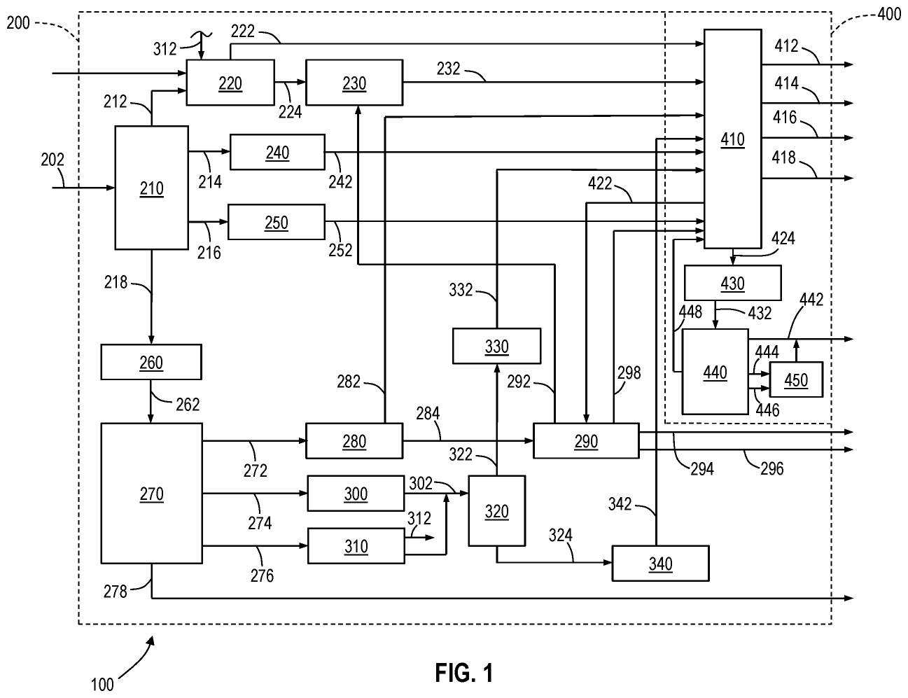

The present disclosure provides a system and method for converting crude oil to light hydrocarbon products useful as a chemical feedstock. An exemplary system 100 is shown in FIG. 1. Unless otherwise indicated, each processing unit of system 100 may be operated under conditions typical for such units. Also, each processing unit of system 100 may include a single unit or multiple sub-units in combination to achieve desired results.

The system 100 of FIG. 1 includes a feed preparation system 200 (shown in phantom) that converts the crude oil feedstock to the chemical feedstock. An exemplary chemical feedstock produced by the feed preparation system 200 may include one or more light hydrocarbon products, such as naphtha, liquefied petroleum gas (LPG), other normal light paraffins (e.g., paraffins of ethane (C2), propane (C3), n-butane (C4), n-pentane (C5), and n-hexane (C6)), and precursors thereof. The chemical feedstock may also contain C10-350° C. boiling range hydrocarbons with a minimum hydrogen content of 14 wt. %. Other materials may be absent or substantially absent from the chemical feedstock, including methane, pyoil, 350+° C. boiling range hydrocarbons, heavy aromatics (e.g., C7+ aromatics), and precursors thereof. For example, the chemical feedstock may contain 0.0 wt. % of each “absent” constituent (e.g., 350+° C. boiling range hydrocarbons) and less than about 1.0 wt. %, 2.0 wt. %, or 3.0 wt. % of each “substantially absent” constituent (e.g., C7+ aromatics). In certain embodiments, the feed preparation system 200 may convert about 75 wt. %, 80 wt. %, 85 wt. %, 90 wt. %, or more of the crude oil feedstock to the chemical feedstock. The feed preparation system 200 is described further in Section 2 below.

The system 100 of FIG. 1 also includes a separate or integrated conversion system 400 (shown in phantom) that converts the chemical feedstock from the feed preparation system 200 to useful hydrocarbon chemicals. Exemplary hydrocarbon chemicals produced by the conversion system 400 include light olefins and di-olefins, such as ethylene, propylene, and/or butadiene and C4 olefins. In certain embodiments, the feed preparation system 200 and the conversion system 400 may cooperate to convert about 60%, 70%, or more of the crude oil feedstock to useful hydrocarbon chemicals. The conversion system 400 is described further in Section 3 below.

The feed preparation system 200 and the conversion system 400 of system 100 may be integrated by sharing a geographical site, power, steam, methane, hydrogen, and/or water, for example. Advantageously, system 100 may minimize the amount of crude oil needed by the feed preparation system 200 while maximizing the production of useful hydrocarbon chemicals by the conversion system 400. Also, system 100 may produce important synergies between the feed preparation system 200 and the conversion system 400, including shared use of light hydrocarbons, methane, and/or hydrogen as well as utility systems, for example.

### 2. Preparation of Chemical Feedstock in the Feed Preparation System

As discussed in Section 1 above, the system 100 includes a feed preparation system 200 that converts the crude oil feedstock to the chemical feedstock. As shown in FIG. 1, the crude oil feedstock is received along conduit 202. The crude oil feedstock may include Arab Extra Light crude oil, Arab Light crude oil, or another suitable crude oil, for example.

The crude oil feedstock in conduit 202 is directed to a crude oil distillation unit (CDU) 210. The CDU 210 of FIG. 1 illustratively separates the crude oil feedstock into at least the following fractions: a gas fraction (e.g., C4 and lighter) along conduit 212; a naphtha fraction along conduit 214; a middle distillate fraction comprising middle-weight hydrocarbons (e.g., C10-350° C. boiling range hydrocarbons) along conduit 216; and a heavy distillate or residue fraction (e.g., fuel oil) along conduit 218.

The gas fraction from conduit 212 of the CDU 210 may be directed to an optional mercaptan sulfur extraction unit (not shown) and a gas concentration unit 220, which may include a C3/C4 splitter or “depropanizer.” An exemplary mercaptan sulfur extraction unit is a Merox™ unit available from UOP LLC, A Honeywell Company, of Des Plaines, Ill., and an exemplary gas concentration unit 220 is UOP's Gas Concentration unit. From the gas concentration unit 220, C3 and lighter constituents continue downstream along conduit 222, while C4 is directed to an isomerization unit 230 along conduit 224. The isomerization unit 230 is described further below.

The isomerization unit 230 produces equilibrium amounts of branched butane (iso-C4) and normal butane (n-C4) in the presence of hydrogen and a fixed-bed catalyst (e.g., a chlorided alumina catalyst). An exemplary isomerization unit 230 is UOP's Butamer™unit. In a conventional refinery, n-C4 is generally fed to the isomerization unit, and the isomerization unit establishes equilibrium by converting the n-C4 to iso-C4. In the present system 100, by contrast, iso-C4 is fed to the isomerization unit 230 along conduit 224, and the isomerization unit 230 establishes equilibrium by converting the iso-C4 to n-C4 along conduit 232. The production of n-C4 by the isomerization unit 230 may be enhanced by removing and recycling any remaining iso-C4 in conduit 232 back into the isomerization unit 230. This recycling process may be repeated until the iso-C4 in conduit 232 is substantially or entirely extinguished.

The naphtha fraction from conduit 214 of the CDU 210 is directed to a naphtha hydrotreating (NHT) unit 240. An exemplary NHT unit 240 is UOP's Naphtha Hydrotreating unit. In the NHT unit 240, the naphtha fraction is combined with hydrogen and passed through a fixed-bed catalyst (e.g., a base metal catalyst) at an elevated temperature to saturate carbon-carbon double bonds and remove heteroatoms (e.g., sulfur and nitrogen). The hydrotreated light distillates from the NHT 240 continue downstream along conduit 242.

The middle distillate fraction from conduit 216 of the CDU 210 is directed to a middle distillate hydrotreating (MDHT) unit 250. An exemplary MDHT unit 250 is UOP's Distillate Unionfining™ unit. In the MDHT unit 250, the middle distillate fraction is combined with hydrogen and passed through a fixed-bed catalyst at an elevated temperature and an elevated pressure to saturate carbon-carbon double bonds as well as aromatics, and remove heteroatoms (e.g., sulfur and nitrogen). To maximize the hydrogen content and minimize the aromatic content of the hydrotreated middle distillates from the MDHT unit 250, the elevated pressure of the MDHT unit 250 may be about 90 bars or more. For example, the elevated pressure of the MDHT unit 250 may be as low as about 90 bars, 100 bars, or 110 bars and as high as about 120 bars, 130 bars, 140 bars, or more, or within any range delimited by any pair of the foregoing values. Also, the catalyst used in the MDHT unit 250 may be a super-high activity Ni/Mo catalyst, such as the KF 860 catalyst available from Albemarle Corp. of the Netherlands. The hydrotreated middle distillates from the MDHT 250 continue downstream along conduit 252. These hydrotreated middle distillates in conduit 252 may have a hydrogen content of about 15 wt. %, 14 wt. %, 13 wt. %, or less, for example.

The heavy distillate or residue fraction from conduit 218 of the CDU 210 is directed to a residue hydrotreating unit 260. An exemplary residue hydrotreating unit 260 is UOP's RCD Unionfining™ unit. In the residue hydrotreating unit 260, the heavy distillate fraction is combined with hydrogen and passed through a series of different fixed-bed catalysts to saturate carbon-carbon double bonds and remove contaminants (e.g., metals and sulfur). The hydrotreated heavy distillates from the residue hydrotreating unit 260 continue downstream along conduit 262.

The hydrotreated heavy distillates in conduit 262 are directed to a cracking unit 270, specifically a fluid catalytic cracking (FCC) unit. An exemplary cracking unit 270 is UOP's PetroFCC™ unit. In the cracking unit 270, the hydrotreated heavy distillates contact a hot fluidized catalyst (e.g., a zeolite catalyst) to vaporize and break down into lighter components. The cracking unit 270 of FIG. 1 illustratively converts the reacted heavy distillate fraction into at least the following fractions: an unsaturated gas fraction along conduit 272; a light hydrocarbon fraction rich in olefins and aromatics along conduit 274; a light cycle oil (LCO) fraction rich in multi-ring aromatics along conduit 276; and a heavy waste fraction (e.g., clarified slurry oil (CSO)) along conduit 278.

The unsaturated gas fraction from conduit 272 of the cracking unit 270 may be directed to a gas concentration unit (not shown) and to an optional mercaptan sulfur extraction unit (not shown), followed by a C3/C4 splitter or “depropanizer” 280. An exemplary mercaptan sulfur extraction unit is UOP's Merox™ Unit, and an exemplary gas concentration unit is UOP's Gas Concentration unit. From the C3/C4 splitter 280, unsaturated C3 and lighter constituents continue downstream along conduit 282, while unsaturated C4 constituents (e.g., mixture of C4 olefins) are directed to a butene processing unit 290 along conduit 284 to produce butene-1. The butene processing unit 290 is described further below.

The butene processing unit 290 may include one or more selective hydrogenation units, catalytic reaction units, and/or separation units. An exemplary butene processing unit 290 includes UOP's Huels Selective Hydrogenation unit, which exposes butadiene and acetylene to hydrogen at mild temperatures and moderate pressures to remove them from the downstream unit feed, and UOP's Ethermax™ unit, which further processes the products from the Huels Selective Hydrogenation unit and catalytically converts iso-butene to methyl tertiary butyl ether (MTBE). The butene processing unit 290 of FIG. 1 illustratively separates the C4 into at least the following fractions: an iso-butane (iso-C4) rich fraction along conduit 292, a MTBE fraction along conduit 294, a butene-1 fraction along conduit 296, and a C4 byproducts fraction (e.g., butene-2, n-C4) along conduit 298. Any iso-butene that is present in conduit 292 may be reacted with hydrogen to saturate carbon-carbon double bonds, eliminate the olefin content, and produce iso-C4, and then the olefin-free iso-C4 may be directed to the above-described isomerization unit 230 for conversion to n-C4.

The light hydrocarbon fraction from conduit 274 of the cracking unit 270, which is rich in olefins and aromatics, is directed to a light distillate or naphtha hydrotreating (NHT) unit 300, which may be similar to the above-described NHT unit 240 associated with the CDU 210 by saturating carbon-carbon double bonds in the olefins and removing heteroatoms. The hydrotreated light fraction from the NHT 300 continues downstream along conduit 302.

The LCO fraction from conduit 276 of the cracking unit 270, which is rich in multi-ring aromatics, is directed to a hydrocracking unit 310. An exemplary hydrocracking unit 310 is UOP's Unicracking™ Process unit. In the hydrocracking unit 310, the multi-ring aromatics in the LCO fraction are broken down into two main fractions, illustratively a light hydrocarbon fraction (e.g., saturated C4 hydrocarbons) and a single-ring aromatic fraction (e.g., aromatic-rich naphtha (C5-C10) hydrocarbons). The light hydrocarbon fraction (e.g., saturated C4 hydrocarbons) is sent to the gas concentration unit 220 along conduit 312, while the single-ring aromatic fraction (e.g., aromatic-rich naphtha) is combined with the hydrotreated light fraction from the NHT 300 in conduit 302.

The combined fractions in conduit 302 are directed to a separation unit 320, which may include a C6−/C7+ splitter. From the separation unit 320, the C6 paraffins and lighter constituents (e.g., C5/C6 constituents) are directed to an isomerization unit 330 along conduit 322, while the C6 naphthene and heavier constituents (e.g., C7+ aromatic constituents) are directed to an aromatic saturation unit 340 along conduit 324. The isomerization unit 330 and the aromatic saturation unit 340 are described further below.

The isomerization unit 330 produces equilibrium amounts of branched C5/C6 and normal C5/C6 (n-C5/C6) in the presence of hydrogen and a fixed-bed catalyst (e.g., a chlorided alumina catalyst). An exemplary isomerization unit 330 is UOP's Penex unit. In a conventional refinery, n-C5/C6 is generally fed to the isomerization unit, and the isomerization unit establishes equilibrium by converting the n-C5/C6 to branched C5/C6. In the present system 100, by contrast, branched C5/C6 is fed to the isomerization unit 330 along conduit 322, and the isomerization unit 330 establishes equilibrium by converting the branched C5/C6 to n-C5/C6 along conduit 332. The production of n-C5/C6 by the isomerization unit 330 may be enhanced by removing and recycling any remaining branched C5/C6 in conduit 332 back into the isomerization unit 330. This recycling process may be repeated until the branched C5/C6 in conduit 332 is substantially or entirely extinguished.

The aromatic saturation unit 340 exposes the C6 naphthene and heavier constituents (e.g., C7+ aromatic constituents) from conduit 324 to hydrogen and a highly active catalyst (e.g., a noble-metal catalyst) at mild temperatures to saturate carbon-carbon double bonds and produce naphthene-rich C7 constituents. An exemplary aromatic saturation unit 340 is UOP's Unisar™ unit. The naphthene-rich C7 constituents from the aromatic saturation unit 340 continue downstream along conduit 342. The naphthene-rich C7 constituents in conduit 342 may make up less than about 1.0 wt. %, 2.0 wt. %, or 3.0 wt. % of the materials fed to the conversion system 400.

### 3. Conversion to Useful Chemicals in the Conversion System

As discussed in Section 1 above, the illustrative system 100 also includes a conversion system 400 that converts the chemical feedstock from the feed preparation system 200 to useful hydrocarbon chemicals. The chemical feedstock from the feed preparation system 200 generally includes light, saturated hydrocarbon products. In the illustrated embodiment of FIG. 1, the chemical feedstock from the feed preparation system 200 includes the following constituents: the C3 and lighter constituents from the gas concentration unit 220 along conduit 222; the n-C4 from the isomerization unit 230 along conduit 232; the hydrotreated light distillates from the NHT 240 along conduit 242; the hydrotreated middle distillates from the MDHT 250 along conduit 252; the unsaturated C3 from the C3/C4 splitter 280 along conduit 282; the C4 byproducts from the butene processing unit 290 along conduit 298; the n-C5/C6 from the isomerization unit 330 along conduit 332; and the naphthene-rich C7 constituents from the aromatic saturation unit 340 along conduit 342.

The conversion system 400 illustratively includes a steam cracker unit 410. In the steam cracker unit 410, the chemical feedstock from the feed preparation system 200 may be diluted with steam and heated in a furnace without oxygen to produce unsaturated hydrocarbons, such as ethylene along conduit 412, propylene along conduit 414, and butadiene along conduit 416. The steam cracker unit 410 may also produce pyrolysis oil (pyoil) along conduit 418. The products produced by the steam cracker unit 410 depend on the composition of the chemical feedstock from the feed preparation system 200, the hydrocarbon-to-steam ratio, the furnace temperature, and the furnace residence time. For example, minimizing the presence of methane (C1), branched light hydrocarbons, 350+° C. boiling range hydrocarbons, heavy aromatics (e.g., C7+ aromatics), and precursors thereof in the chemical feedstock from the feed preparation system 200 may minimize the production of methane and pyoil in the steam cracker unit 410 along conduit 418.

The steam cracker unit 410 may also produce a butene raffinate, which may be returned to the above-described butene processing unit 290 along conduit 422.

The steam cracker unit 410 may further produce pyrolysis gasoline (pygas), which may be directed to a pygas hydrotreatment unit 430 along conduit 424. An exemplary pygas hydrotreatment unit 430 is UOP's Pygas Hydrotreating Process unit. In the pygas hydrotreatment unit 430, the pygas may be combined with hydrogen and passed through a fixed-bed catalyst at an elevated temperature to saturate carbon-carbon double bonds and remove heteroatoms (e.g., sulfur). The hydrotreated pygas from the pygas hydrotreatment unit 430 may continue downstream along conduit 432.

The hydrotreated pygas in conduit 432 may be directed to an aromatics extraction unit 440. An exemplary aromatics extraction unit 440 is UOP's Sulfolane™ unit. The aromatics extraction unit 440 may subject the hydrotreated pygas to extractive distillation to extract benzene, toluene and C8+ aromatics. The aromatic extract is further separated to benzene along conduit 442, toluene along conduit 444, and C8+ aromatics along conduit 446. The raffinate from the aromatics extraction unit 440 may be returned to the steam cracker unit 410 along conduit 448.

The toluene in conduit 444 and the C8+ aromatics in conduit 446 may be directed to a dealkylation unit 450, as shown in FIG. 1. An exemplary dealkylation unit 450 is UOP's Thermal Dealkylation (THDA) unit. In the dealkylation unit 450, radicals may be stripped from the toluene and C8+ aromatics to produce benzene, which may be combined with the extracted benzene in conduit 442. Alternatively, the toluene in conduit 444 and the C8+ aromatics in conduit 446 may be output directly from the aromatics extraction unit 440 and sold without being directed to the dealkylation unit 450.

### 4. System Mass Balance

An exemplary mass balance for the illustrative system 100 is presented in Error! Not a valid bookmark self-reference. below. As shown below, about 60% of the crude oil feedstock may be converted to light olefins, including ethylene, propylene, and butadiene.

TABLE 1

| | Materials | Range | Example |
|---|---|---|---|
| Feeds | Crude oil | 92-96% | 94% |
| | Natural gas | 2-6% | 4% |
| | Methanol | 0-4% | 2% |
| | Total | 100% | 100% |
| Products | Ethylene | 27-31% | 29% |
| | Propylene | 20-24% | 22% |
| | Butadiene | 3-7% | 5% |
| | Butene-1 | 1-5% | 3% |
| | MTBE | 4-8% | 6% |
| | Benzene | 7-11% | 9% |
| | Toluene | 0-4% | 2% |
| | Other (hydrogen, methane, pyoil, | 22-26% | 24% |
| | solids, etc.) | | |
| | Total | 100% | 100% |

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A system for converting a crude oil feedstock to a chemical feedstock for a steam cracker unit, the system comprising:
a distillation unit that separates the crude oil feedstock into at least:
a hydrocarbon gas fraction;
a naphtha fraction;
a middle distillate fraction; and
a residue fraction;
a first isomerization unit having a first input that receives the hydrocarbon gas fraction from the distillation unit and a first output, the first isomerization unit establishing equilibrium by converting a branched butane in the first input to a normal butane in the first output;
a naphtha hydrotreating unit that receives the naphtha fraction from the distillation unit, the naphtha hydrotreating unit saturating the naphtha fraction;
a middle distillate hydrotreating unit that receives the middle distillate fraction from the distillation unit, the middle distillate hydrotreating unit saturating the middle distillate fraction;
a residue hydrotreating unit that receives the residue fraction from the distillation unit, the residue hydrotreating unit saturating the residue fraction;
a cracking unit that receives the saturated residue fraction from the residue hydrotreating unit, the cracking unit converting the saturated residue fraction into at least:
an unsaturated gas fraction;
a light hydrocarbon fraction rich in olefins and aromatics;
a light cycle oil fraction rich in rich in multi-ring aromatics; and
a heavy waste fraction;
a butene processing unit that receives the unsaturated gas fraction from the cracking unit, the butene processing unit producing at least an iso-butane rich fraction;
a second isomerization unit having a second input that receives the light hydrocarbon fraction from the cracking unit and a second output, the second isomerization unit establishing equilibrium by converting a branched pentane and a branched hexane in the second input to a normal pentane and a normal hexane in the second output; and
an aromatic saturation unit that receives the light cycle oil fraction from the cracking unit, the aromatic saturation unit saturating the light cycle oil fraction and producing a naphthene-rich fraction; and
wherein the chemical feedstock to the steam cracker unit includes at least:
the first output from the first isomerization unit;
the saturated naphtha fraction from the naphtha hydrotreating unit;
the saturated middle distillate fraction from the middle distillate hydrotreating unit;
the second output from the second isomerization unit; and
the naphthene-rich fraction from the aromatic saturation unit.

2. The system of claim 1, wherein the first and second isomerization units use chlorided alumina catalysts.

3. The system of claim 1, wherein the butene processing unit directs the iso-butane rich fraction to the first isomerization unit.

4. The system of claim 1, wherein the naphthene-rich fraction from the aromatic saturation unit comprises less than about 3.0 wt. % of the chemical feedstock to the steam cracker unit.

5. The system of claim 1, wherein the middle distillate hydrotreating unit operates at a pressure of about 90 bars or more.

6. The system of claim 1, wherein the saturated middle distillate fraction from the middle distillate hydrotreating unit has a hydrogen content of at least about 14 wt. %.

7. The system of claim 1, wherein the middle distillate hydrotreating unit uses a Ni—Mo catalyst.

8. The system of claim 1, further comprising a first splitter downstream of the distillation unit and upstream of the first isomerization unit, the first splitter separating the hydrocarbon gas fraction from the distillation unit into at least:
a first fraction comprising saturated C3 and lighter constituents; and
a second fraction comprising the branched butane that is directed to the first isomerization unit;
wherein the first fraction is directed from the first splitter to the steam cracker unit such that the chemical feedstock to the steam cracker unit further includes the first fraction.

9. The system of claim 1, further comprising a second splitter downstream of the cracking unit and upstream of the butene processing unit, the second splitter separating the unsaturated gas fraction from the cracking unit into at least:
a first fraction comprising unsaturated C3 and lighter constituents; and
a second fraction comprising unsaturated C4 constituents;
wherein the first fraction is directed from the second splitter to the steam cracker unit such that the chemical feedstock to the steam cracker unit further includes the first fraction; and
wherein the second fraction is directed from the second splitter to the butene processing unit.

10. The system of claim 1, further comprising a third splitter downstream of the cracking unit, the third splitter receiving both the light hydrocarbon fraction and the light cycle oil fraction from the cracking unit.

11. The system of claim 10, wherein the third splitter is upstream of both the second isomerization unit and the aromatic saturation unit.

12. The system of claim 11, further comprising a hydrotreating unit downstream of the cracking unit and upstream of the third splitter, the hydrotreating unit saturating the olefins in the light hydrocarbon fraction before reaching the third splitter and the second isomerization unit.

13. The system of claim 11, further comprising a hydrocracking unit downstream of the cracking unit and upstream of the third splitter, the hydrocracking unit breaking down the multi-ring aromatics in the light cycle oil fraction before reaching the third splitter and the aromatic saturation unit.

14. A method for converting a crude oil feedstock to a chemical feedstock for a steam cracker unit, the method comprising the steps of:
separating the crude oil feedstock into at least:
a hydrocarbon gas fraction;
a naphtha fraction;
a middle distillate fraction; and
a residue fraction;
converting a branched butane in the hydrocarbon gas fraction to a normal butane in a first isomerization unit by establishing equilibrium in the first isomerization unit;
saturating the naphtha fraction in a naphtha hydrotreating unit;
saturating the middle distillate fraction in a middle distillate hydrotreating unit;
saturating the residue fraction in a residue hydrotreating unit;
converting the saturated residue fraction in a cracking unit into at least:
an unsaturated gas fraction;
a light hydrocarbon fraction rich in olefins and aromatics;
a light cycle oil fraction rich in rich in multi-ring aromatics; and
a heavy waste fraction;
converting the unsaturated gas fraction into at least an iso-butane rich fraction in a butene processing unit;
converting a branched pentane and a branched hexane in the light hydrocarbon fraction to a normal pentane and a normal hexane in a second isomerization unit by establishing equilibrium in the second isomerization unit;
saturating the light cycle oil fraction in an aromatic saturation unit to produce a naphthene-rich fraction; and
directing the chemical feedstock to the steam cracker unit, wherein the chemical feedstock includes at least:
the normal butane from the first isomerization unit;
the saturated naphtha fraction from the naphtha hydrotreating unit;
the saturated middle distillate fraction from the middle distillate hydrotreating unit;
the normal pentane and the normal hexane from the second isomerization unit; and
the naphthene-rich fraction from the aromatic saturation unit.

15. The method of claim 14, further comprising the step of producing at least one useful chemical in the steam cracker unit, the at least one useful chemical selected from the group consisting of: ethylene, propylene, and butadiene.

16. The method of claim 14, further comprising the step of directing the iso-butane rich fraction from the butene processing unit to the first isomerization unit.

17. The method of claim 16, further comprising the step of saturating iso-butene to iso-butane before the first isomerization unit.

18. The method of claim 14, wherein saturating the middle distillate fraction in the middle distillate hydrotreating unit occurs at a pressure of about 90 bars or more.
